(19) United States
(12) Patent Application Publication
(10) Pub. No.: US 2020/0254170 A1
(43) Pub. Date: Aug. 13, 2020

(54) BLOOD FILTRATION CARTRIDGE END CAP

(71) Applicants: Vincent CAPPONI, Monmouth Junction, NJ (US); Paul ROBERTSON, Raleigh, NC (US); CytoSorbents Corporation, Monmouth Junction, NJ (US)

(72) Inventors: Vincent CAPPONI, Monmouth Junction, NJ (US); Paul ROBERTSON, Raleigh, NC (US)

(21) Appl. No.: 16/639,371

(22) PCT Filed: Aug. 21, 2018
(86) PCT No.: PCT/US18/47241

§ 371 Date: Feb. 14, 2020

Related U.S. Application Data
(60) Provisional application No. 62/548,642, filed on Aug. 22, 2017.

(51) Int. Cl. A61M 1/34; A61M 1/36

(52) U.S. Cl. CPC A61M 1/3486; A61M 2206/20; A61M 2205/75; A61M 1/3679

(57) ABSTRACT

The invention relates to a removable cap for a blood filter cartridge useful in facilitating dispersal of whole blood in the cartridge. The cartridge has a removable cap on at least one and preferably both of its input and output ends. The caps comprise an annular flange means having attached thereto an outer annular wall and an annular inner wall. The annular inner wall defining a cavity covered by a conical, concave filter mesh. The end cap also comprises a channel means positioned under the cavity and a part of said flange means, for reception of any fluid which passes through said concave filter mesh.

### FIELD OF THE INVENTION

The invention relates to a removable cap for a blood filter cartridge useful in facilitating dispersal of whole blood in the cartridge. The cartridge has a removable cap on at least one and preferably both of its input and output ends.

### BACKGROUND AND PRIOR ART

Blood filter cartridges are well known. See, e.g., U.S. Pat. No. 6,217,540 to Yazawa, et al., incorporated by reference, which shows one variation. In general, such cartridges have a chamber, and a pair of “caps,” positioned at each end of the chamber, one of which includes an inlet port and the other an outlet port for passage of the fluid through the camber. See, e.g., U.S. Patent Publication 2018/0200651, incorporated by reference. It has been suggested that one method of separating cells from other blood components is by use of blood filter cartridges which include therein a hemocompatible polymer which is able to retain the desired cells thereon.

A problem in blood filtration using cartridges of this type is that the sample, especially whole blood, tends not to readily disperse within the cartridge. Rather, at times the blood collects directly under the inlet port and clots form due to operation of the complex phenomenon known as the “coagulation cascade”. If this occurs, filtration slows down, is less than optimal, or fails.

In accordance with the present invention, the chamber of the blood filtration cartridge contains a hemocompatible polymer to which a molecule specific for a target of interest (e.g., an antibody specific for a receptor or other cell surface marker of the desired type of cell), maybe, but is not necessarily attached. In operation, the sample passes through the cartridge via the inlet port, and any target cells of the desired type bind to the molecule or are adsorbed by the polymer remain within the cartridge. The undesired blood components traverse the cartridge and pass through the outlet port. A class of hemocompatible polymers which can function in such a system are polymers of the general type disclosed in various patents owned by Cytosorbents Corporation, e.g., U.S. Pat. Nos. 7,875,182 and 9,604,196 both incorporated by reference.

The present invention preferably also provides an end cap at the inlet port and preferably an end cap at the outlet port as well, each of which is fitted with a hemocompatible mesh filter to facilitate dispersion of the fluid through the filter cartridge.

### BRIEF DESCRIPTION OF THE FIGURES

FIGS. 1a and 1b show machined and molded plane views of embodiments of the end cap of the invention.

FIGS. 2a and 2b show cutaway views of machined and molded embodiments of the invention, to illustrate particular features.

FIGS. 3a and 3b show additional cutaway views of machined and molded embodiments of the invention, to show additional features of the invention.

FIG. 4 is an additional cutaway view of a machined embodiment of the invention.

FIG. 5 is an additional cutaway view of a molded embodiment of the invention.

### DETAILED DESCRIPTION OF PREFERRED EMBODIMENTS

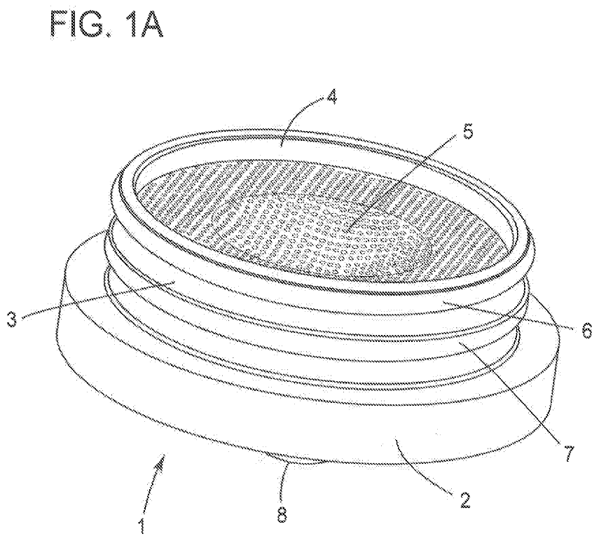

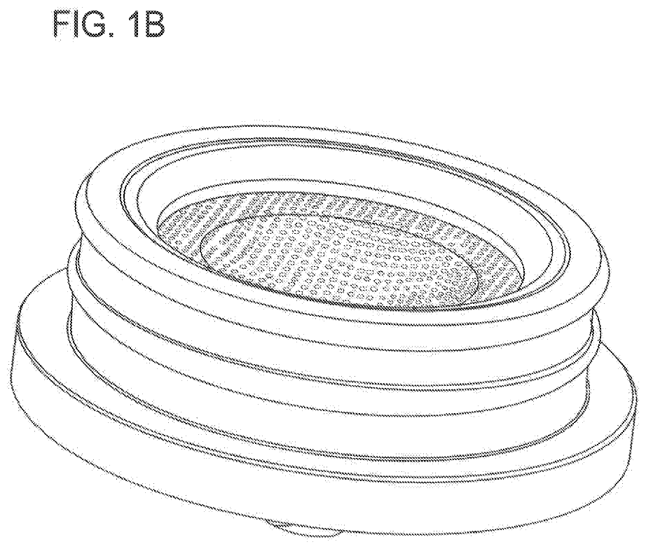

FIGS. 1a and 1b show an embodiment of the invention. FIG. 1a shows a machined, and FIG. 1b shows a molded form of the invention.

In each embodiment, end cap “I” comprises an annular flange “2,” which engages the aforementioned filtration cartridge.

Attached to flange “2” is an annular outer wall “3,” and an annular inner wall “4,” in concentric form. At the center of the inner wall is a cavity, covered by a concave filter mesh “5,” which assumes a conical form. This concave filter mesh is flexibly attached to the inner wall, and moves upon application of pressure from fluid applied to it. The concave filter mesh is attached to the inner wall via, e.g., an adhesive, or by solvent bonding, ultrasonic bonding, spin welding, insert molding, or other art recognized techniques.

In the embodiment shown in FIGS. 1a and 1b, the outer wall “2” has a ribbed configuration, characterized by repeating recessed and protruding concentric rings (6” and “7”, respectively). The outer ring has a diameter less than that of the flange, and may be molded to the flange in a one piece construction, or attached to it via any of the attachment methodologies described supra.

Barely visible in FIGS. 1a and 1b, is a channel means “8,” which is positioned below said cavity and is a part of said annular flange means, which permits filtered fluid to pass to the cartridge, not shown in the figures.

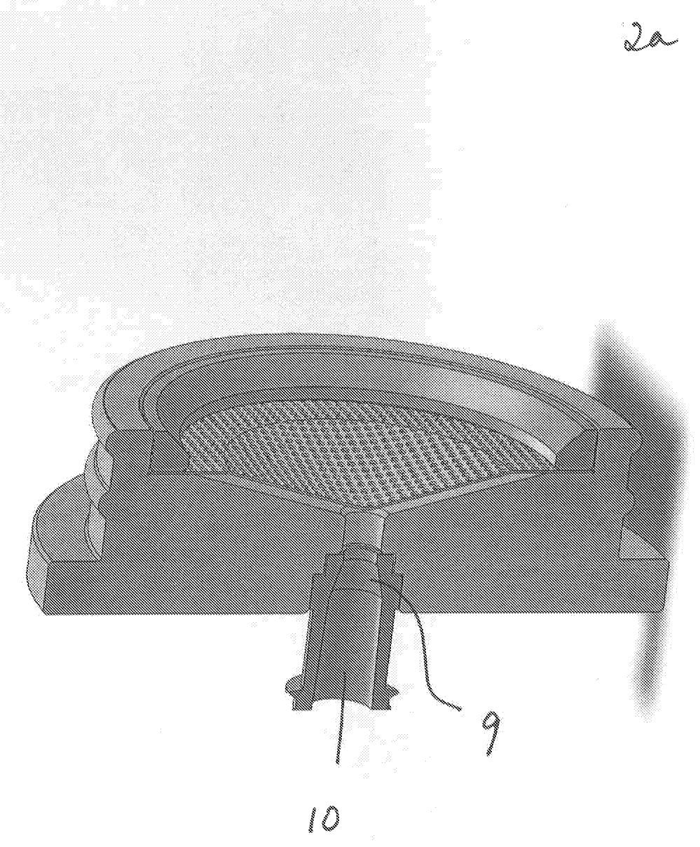

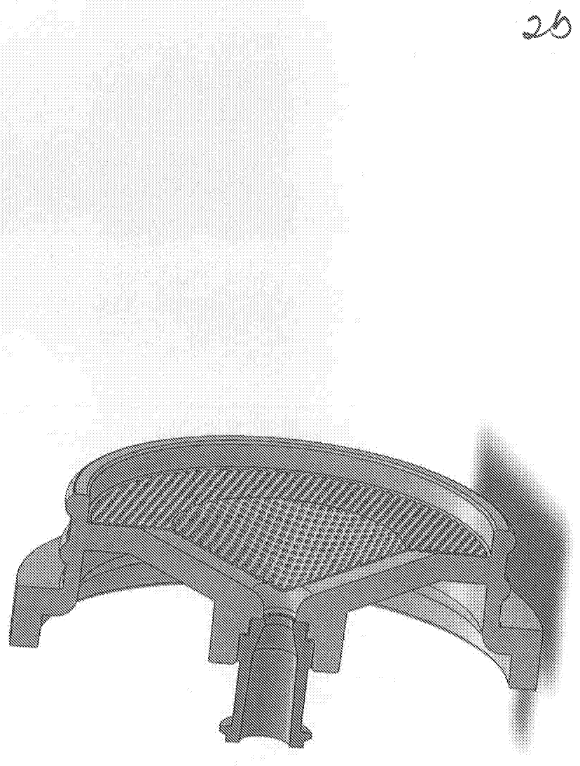

FIGS. 2a and 2b are also machined (FIG. 2a) and molded (FIG. 2b) embodiments of the end cap of the invention. These figures show a cut view of the embodiment in FIGS. 1a and 1b, so as to show the channel or transport means more clearly. In this embodiment, luer “9” can be seen. While “9” is a female luer, a male or a female luer may be used.

The luer extends from the from the mesh filter to a channel means “10.” When the cartridge is not in use, the luer is covered. Further, while not shown, the luer may include a horse barb connection.

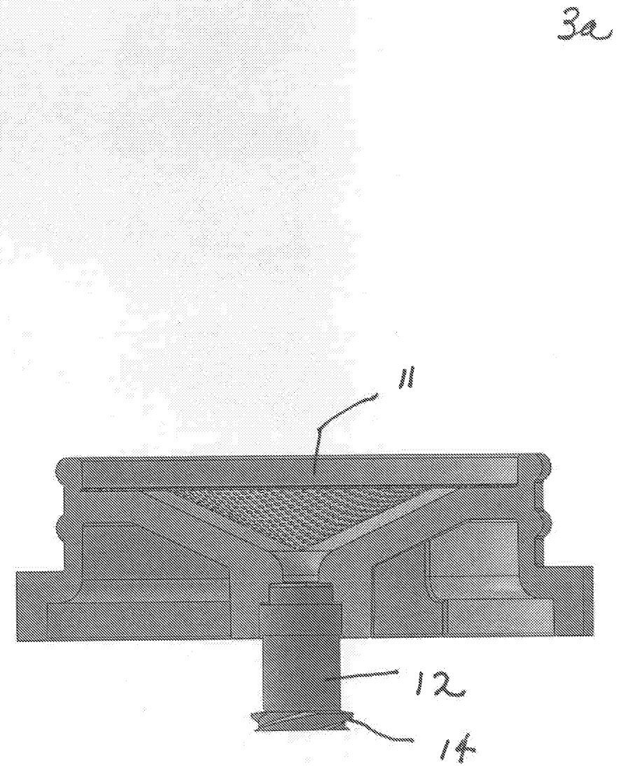

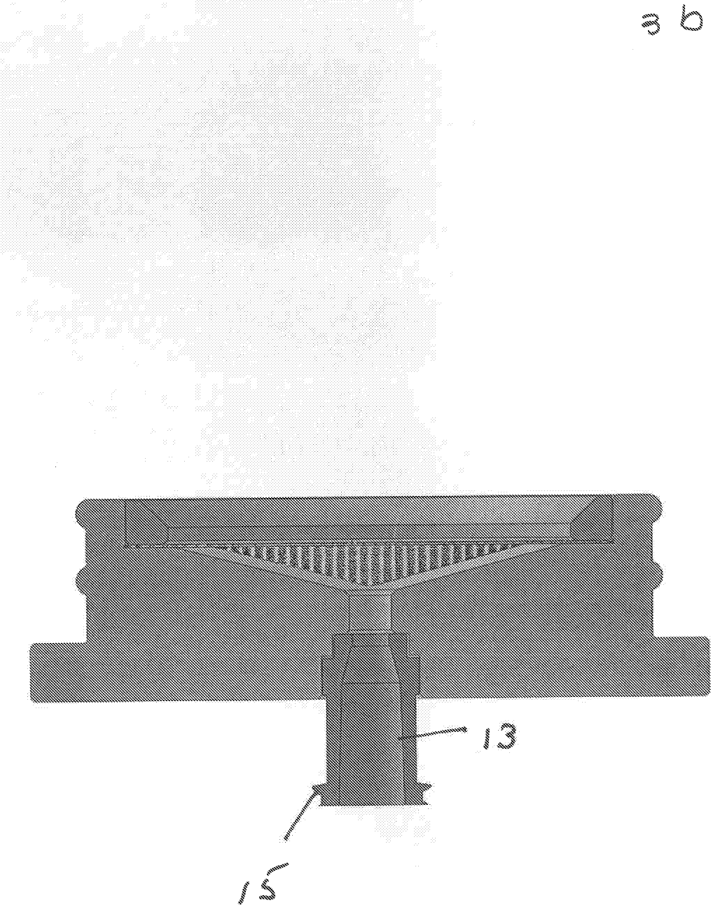

FIGS. 3a and 3b also show machined (FIG. 3a) and molded (FIG. 3b), embodiments of the invention, with a circular horizontal covering means “11,” which engages the outer wall and fits said cavity for when the cartridge is not in use. These side views also show alternate forms of the luer “9,” shown as “12” and “13,” as well as means “14” and “15” for joining the cap to the channel means discussed supra.

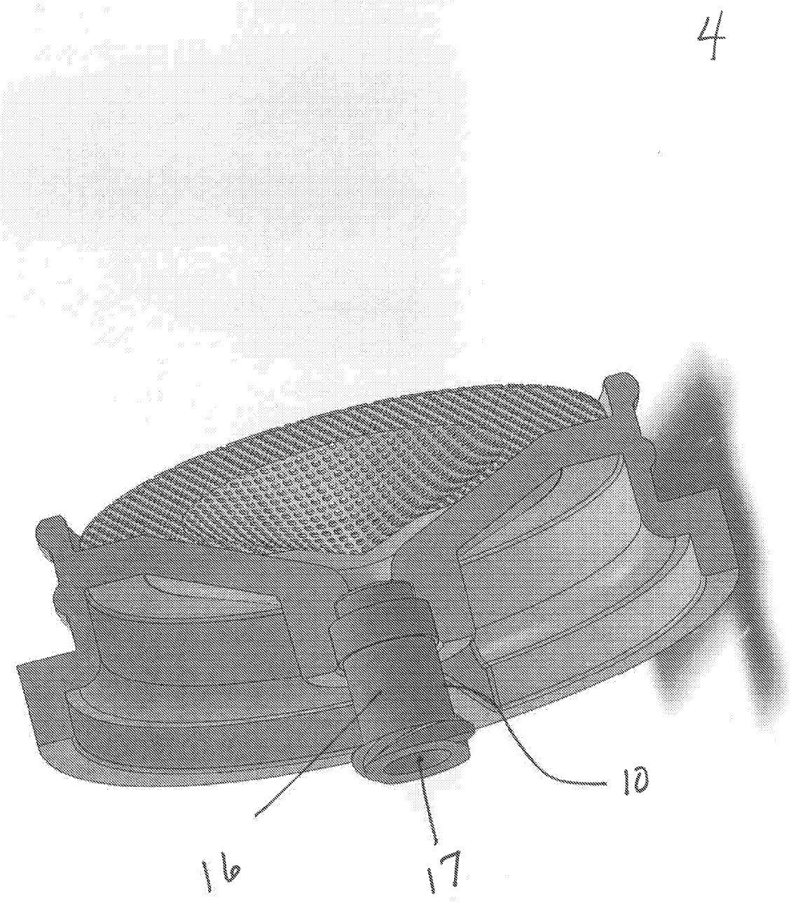

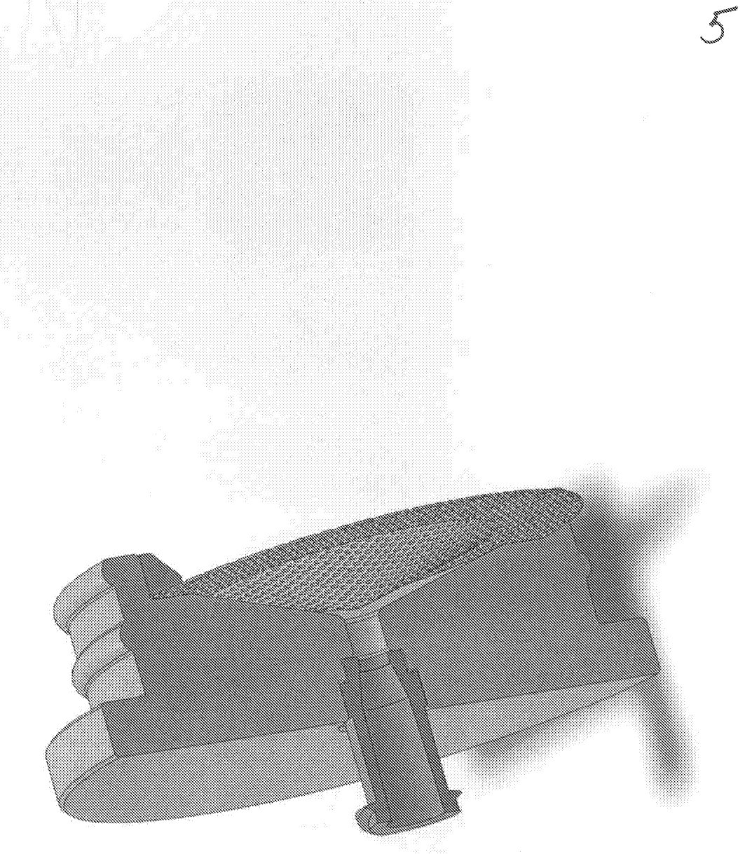

FIG. 4 shows a cut view of the cap showing channel means “10” more clearly. One sees a cylindrical wall “16,” and hollow cavity “17.”

The mesh preferably comprises uniform openings with a diameter ranging from about 5μ to about 150μ, and preferably 50-100μ. Prior to use, the mesh should be sterilized via means known to the art (e.g., gamma radiation, ethylene oxide (ETO), E Beam, or steam).

The luer includes a cavity which extends to the conical concave filter mesh, and has a covering means, noted supra, for when the cartridge of which the end cap is a part, is not being used. The luer preferably comprises a hose barb connection, or other means for connecting tubing to the luer. The connection of luer and tubing assists any entering fluid to disperse uniformly, so as to inhibit or to reduce the likelihood of problems with fluid passage (e.g., blood clotting).

Both the inlet cap and outlet cap of the cylinder are preferably the same. As will be appreciated, the mesh in the outlet cap will be oriented in the opposite direction to fluid flow of the mesh on the inlet cap and, if the outlet cap is oscillated in response to fluid flow pressure, it will assist in regulating the flow and/or dispersion of the fluid within the cartridge. Although the pressure on the fluid will permit outlet flow, preferably the mesh provides a degree of resistance which assists in helping to maximize the adsorbent capture of the desired cells specific for the hemocompatible polymer molecules (e.g., antibodies) attached to the polymer.

As will be understood by a person of ordinary skill in this field, the size of the cartridge will vary, and the diameter of the in (out)put caps, and their size will depend upon the volume of the cartridge with which they are being used. Caps for, e.g., a 60 ml cartridge, are smaller than those for a 140 ml cartridge.

Other embodiments of the invention will be readily apparent to those of ordinary skill in the art.

The terms and expression which have been employed are used as terms of description and not of limitation, and there is no intention in the use of such terms and expression of excluding any equivalents of the features shown and described or portions thereof, it being recognized that various modifications are possible within the scope of the invention.

1. An end cap for use in a blood filter device, comprising an annular flange means having attached thereto an outer annular wall and an annular inner wall, said annular inner wall defining a cavity, said cavity covered by a conical, concave filter mesh, said end cap further comprising a channel means positioned under said cavity and a part of said flange means, for reception of any fluid which passes through said concave filter mesh.

2. The end cap of claim 1, wherein said conical concave filter mesh is flexibly attached to said annular inner wall.

3. The end cap of claim 2, wherein said conical concave filter mesh has been flexibly attached to said inner wall via an adhesive, solvent bonding, ultrasonic bonding, spin welding, or insert molding.

4. The end cap of claim, wherein said outer annular wall comprises a ribbed configuration.

5. The end cap of claim 1, wherein said channel means comprises a luer.

6. The end cap of claim 1, further comprising a horizontal covering means which engages said cavity and said outer wall.

7. The end cap of claim 1, wherein said conical concave filter mesh comprises openings with a diameter of from about 5μ to about 150μ.

8. The end cap of claim 7, wherein said openings have a diameter of from about 50μ to about 100μ.

9. The end cap of claim 1, wherein said conical concave filter mesh has been sterilized.

10. The end cap of claim 1, further comprising a hose barb attached to said luer, and tubing means attached thereto.

11. A filter means comprising a cylinder having two end openings which contains a filter material and comprises an end cap of claim 1 at each end openings, wherein said end caps are positioned diametrically to each other.
